(19) United States
(12) Patent Application Publication
(10) Pub. No.: US 2019/0276857 A1
(43) Pub. Date: Sep. 12, 2019

(54) METHOD FOR SECOND GENERATION ETHANOL PRODUCTION FROM LIGNOCELLULOSIC BIOMASS

(71) Applicant: Indian Oil Corporation Limited, Bandra (East) (IN)

(72) Inventors: Ajay Kumar SHARMA, Faridabad (IN); Manas Ranjan SWAIN, Faridabad (IN); Ajit SINGH, Faridabad (IN); Anshu Shankar MATHUR, Faridabad (IN); Ravi Prakash GUPTA, Faridabad (IN); Deepak TULI, Faridabad (IN); Suresh Kumar PURI, Faridabad (IN); Sankara Sri Venkata RAMAKUMAR, Faridabad (IN)

(21) Appl. No.: 16/351,045

(22) Filed: Mar. 12, 2019

(30) Foreign Application Priority Data
Mar. 12, 2018 (IN) 201821008982

(51) Int. Cl. C12P 7/14; C12P 7/06

(52) U.S. Cl. CPC C12P 7/14; C12P 7/065

(57) ABSTRACT

The present invention relates to a process for production of ethanol from lignocellulosic biomass via modified simultaneous saccharification and co-fermentation (SSCF). In the present invention, enzymatic hydrolysis is preceded by mainly C5 sugar fermentation and low enzymatic hydrolysis and succeeds by mainly C6 sugar fermentation at different temperature and duration. This resulted into reduction in enzyme dosage and process time with increase in ethanol yield from acid pretreated biomass.

### FIELD OF THE INVENTION

The present invention relates to a process for production of ethanol from lignocellulosic biomass.

### BACKGROUND OF THE INVENTION

Simultaneous Saccharification fermentation/co-fermentation (SSF/SSCF) removes sugar inhibition on enzymatic hydrolysis thus increases the hydrolysis sugar yield and reduces contamination risk. Moreover, SSF/SSCF reduces the overall reaction time and reactor volume (Kristensen et al., 2009). SSF/SSCF sacrifices the optimal conditions for both enzymatic hydrolysis and fermentation. Typically enzymatic hydrolysis and fermentation in SSF system the temperature is kept at 37-42° C. as a compromise for better enzymatic hydrolysis and fermentation (Dien et al., 2003b). In addition, SSF/SSCF introduces a new inhibitor (ethanol) for enzymatic hydrolysis. But the inhibitory effect from ethanol is much lower compared to cellobiose or glucose (Taherzadeh & Karimi, 2007).

The major advantage of Separate Hydrolysis and fermentation/co-fermentation (SHF/SHCF) compared to SSF/SSCF is that enzymatic hydrolysis and fermentation can be carried out at their own optimal conditions (Taherzadeh & Karimi, 2007). However, enzymes during hydrolysis is easily inhibited by its end-products (sugars), especially during high solid loading enzymatic hydrolysis (Kristensen et al., 2009; Philippidis & Smith, 1995), which led to sluggish hydrolysis and resulted into enhanced hydrolysis time and high enzyme loading to achieve high sugar conversions. Another problem of this process is the high risk of contamination during enzymatic hydrolysis due to the long reaction time and high sugar concentrations (Taherzadeh & Karimi, 2007). Enzymatic hydrolysis is the limiting step for SHF, which determines the overall ethanol yield (Lau & Dale, 2009).

US 20060014260 disclose a simultaneous saccharification and fermentation (SSF) process for the bioconversion of cellulose into ethanol. It discloses that the reaction mixture comprises of slurry comprising cellulosic substrate, an enzyme and a fermentation agent. The reaction mixture is treated at a temperature between about 30° C. and 48° C. and a pH between about 4.0 and 6.0, along with agitation for a period of about 30 minutes to several hours or days.

US 20100268000 A1 discloses method for producing one or more fermentation end-products by fermenting a lignocellulosic biomass comprising hexose and pentose saccharides. Further it discloses using Saccharomyces cerevisiae along with fermentation medium supplement selected from the group consisting of a fatty acid, a surfactant, a chelating agent, vitamins, minerals, pH modifiers, yeast extract, and salts such as ammonium salts and salts of magnesium.

### SUMMARY OF THE INVENTION

In present invention of modified SSCF process, enzymatic hydrolysis is preceded by mainly C5 sugar fermentation and low enzymatic hydrolysis and succeeds by mainly C6 sugar fermentation at different temperature and duration. This resulted into higher ethanol titer at short time of combined hydrolysis and fermentation. Present invention is advantageous over conventional SSCF because initial free sugars and oligosaccharides in the pretreated biomass was targeted for fermentation which reduces the enzymatic feedback inhibition and optimum temperature were used for hydrolysis and fermentation, which resulted into higher ethanol yield at low dose of enzyme at short time interval.

In conventional fermentation practice glucose concentration is always higher than xylose concentration. So in this situation, yeast mostly prefers glucose fermentation which ultimately reduces the xylose fermentation efficiency and prolongs the xylose fermentation time.

In the present study free xylose and other oligosaccharides are targeted initial stage of fermentation which ultimately reduces enzymatic feedback inhibition. Due to which enzyme concentration was reduced to half than the conventional practice and further reduced fermentation time to one third and 30 h less than separate hydrolysis and fermentation (SHF) and conventional SSCF, respectively. This makes the process more acceptable for commercial practice.

Accordingly, present invention provides a process for production of ethanol from a lignocellulosic biomass comprising;
adding slurry of a pre-treated lignocellulosic biomass comprising C5 and C6 sugars in a fermenter system;
(ii) selectively fermenting mainly C5 sugars by incubating the pretreated lignocellulosic biomass with a cellulase enzyme, co-fermenting microorganism and nutrient to obtain ethanol;
(iii) hydrolysing by heating the fermenter system to 48-55° C. for a period of 18 to 24 hours;
(iv) allowing the fermenter system to cool to a temperature of 35-37° C.; and
(v) selectively fermenting C6 sugars by inoculating the system with a second dose of co-fermenting microorganism to obtain ethanol.

In one of the feature, present invention provides a process for production of ethanol from a lignocellulosic biomass comprising;
adding whole slurry of an acid pre-treated lignocellulosic biomass comprising C5 and C6 sugars in a fermenter system;
(ii) selectively fermenting mainly C5 sugars by incubating the pretreated lignocellulosic biomass with a cellulase enzyme, co-fermenting microorganism and nutrient to obtain ethanol;
(iii) hydrolysing by heating the fermenter system to 48-55° C. for a period of 18 to 24 hours;
(iv) allowing the fermenter system to cool to a temperature of 35-37° C.; and
(v) selectively fermenting C6 sugars by inoculating the system with a second dose of co-fermenting microorganism to obtain ethanol.

In one of the feature of the present invention, the fermentation of C5 sugar is carried out at temperature in the range of 30° C.-35° C. for 16-20 hours or any other temperature which favors fermentation over hydrolysis, when the xylose concentration is reduced to 6-7 g/l in fermentation broth the temperature of process is increased to 33 and 35° C. gradually and incubated at 2 h in each temperature for better hydrolysis and fermentation.

In another feature of the present invention, the fermentation of C6 sugar is carried out at temperature in the range of 35 to 37° C. for 6 to 10 hours or any other temperature which favors fermentation over hydrolysis.

In yet another feature of the present invention, the pre-treated biomass slurry is added in the fermenter system of step (i) without any detoxification.

In still another feature of the present invention, the process for production of ethanol from a lignocellulosic biomass additionally comprising adjusting pH of the slurry of step (i) to 5-5.5 with a pH adjuster.

In yet another feature of the present invention, the pH adjuster is selected from aqueous ammonium hydroxide, NaOH, KOH, and CaCO₃ or substance which is alkaline in nature and increases pH.

In still another feature of the present invention, the nutrient is ammonium sulphate, MgSO₄ or any other magnesium or ammonium salts.

In still another feature of the present invention, the cellulase enzyme is from fungal origin, β-glucosidase along with other accessory enzyme, wherein:
(i) the cellulase enzyme of fungal origin is composed of Cellobiohydrolase I and II; and
(ii) the other accessory enzymes is selected from xylanase, β-xyloxidase, arabinofuranosidase, and pectinse.

In yet another feature of the present invention, the co-fermenting (C6 and C5 sugar) microorganism is selected from Saccharomyces cerevisiae, Pichia sp., Candida sp., and E. coli or any ethanogenic co-fermenting microorganism.

In yet another feature of the present invention, the C5 sugar is selected from xylose and C6 sugar is selected from glucose.

In yet another feature of the present invention, the fermentation of C6 sugar is stopped after 6 to 10 hours of fermentation.

In still another feature of the present invention, optionally other nutrient is used to enhance the final ethanol concentration and the other nutrient is selected from yeast extract, peptone and ammonium sulphate or any other nitrogen source for microorganism.

In still another feature of the present invention, the lignocellulosic biomass is selected from straw, wheat straw, sugarcane bagasse, cotton stalk, barley stalk, bamboo or any agriculture residues which contain cellulose or hemicellulose or both.

In one of the feature, present invention provides a process for production of ethanol from a lignocellulosic biomass comprising:
(i) adding a pre-treated biomass slurry comprising C5 and C6 sugars without any detoxification in a fermenter system;
(ii) adjusting pH of the slurry of step (i) to 5-5.5 with aqueous ammonium solution to obtain a pH adjusted slurry;
(iii) fortifying the pH adjusted slurry with MgSO₄ in amount of 3-5 g/l, along with cellulase enzyme and co-fermenting microorganism;
(iv) adding water to the slurry of step (iii) to maintain 5 to 20 weight % Total solid (TS) in the slurry;
(v) incubating the slurry of step (iv) at 30° C.-35° C. for 16-20 hours for a selectively fermenting C5 sugars with 200-250 rpm when free xylose in the slurry to comes down to 6-7 g/l from 30 to 35 g/l;
(vi) hydrolysing by heating the fermenter system to 48-55° C. at ramping of 3 to 4° C. per 20-25 minutes, and then the process is allowed to maintain the temperature 48° C.-55° C. for 18-24 hours;
(vii) allowing the fermenter system to cool down to a temperature 35-37° C.; and
(viii) selectively fermenting C6 sugars by inoculating the system with a second dose of co-fermenting microorganism to obtain ethanol.

In yet another feature, present invention provides a process for production of ethanol from a lignocellulosic biomass comprising:
(i) adding a pre-treated biomass slurry comprising C5 and C6 sugars without any detoxification in a fermenter system;
(ii) adjusting pH of the slurry of step (i) to 5-5.5 with aqueous ammonium solution to obtain a pH adjusted slurry;
(iii) fortifying the pH adjusted slurry with MgSO₄ in amount of 3-5 g/1, along with cellulase enzyme and co-fermenting microorganism;
(iv) adding water to the slurry of step (iii) to maintain desired Total solid (TS) in the slurry;
(iv) incubating the slurry of step (iv) at 30° C.-35° C. for 16-20 hours for a selectively fermenting C5 sugars with 200-250 rpm when free xylose in the slurry to comes down to 6-7 g/l from 30 to 35 g/1; while temperatures increasing, fermentor is hold at 30 and 33° C. for 2 hours to increase rate of hydrolysis and fermentation;
(v) hydrolysing by heating the fermenter system to 48-55° C. at ramping of 3 to 4° C. per 20-25 minutes, and then the process is allowed to maintain the temperature 48° C.-55° C. for 18-24 hours;
(iv) allowing the fermenter system to cool down to a temperature 35-37° C.; and
(vi) selectively fermenting C6 sugars by inoculating the system with a second dose of co-fermenting microorganism to obtain ethanol.

### BRIEF DESCRIPTION OF DRAWINGS

FIG. 1 illustrates flowchart depicting a process for production of ethanol from a lignocellulosic biomass according to an embodiment of the present disclosure.

FIG. 2 illustrates results of invented modified SSCF using Co-fermenting S. cerevisiae and commercial enzyme (3.3 FPU/TS);

FIG. 3 illustrates results of conventional SSCF using S. cerevisiae and commercial enzyme (7 FPU/TS); and

FIG. 4 shows illustration of ethanol yield (%) of the modified SSCF process and conventional SSCF process for ethanol production

### DETAILED DESCRIPTION OF THE INVENTION

While the invention is susceptible to various modifications and alternative forms, specific embodiment thereof will be described in detail below. It should be understood, however that it is not intended to limit the invention to the particular forms disclosed, but on the contrary, the invention is to cover all modifications, equivalents, and alternative falling within the scope of the invention as defined by the appended claims.

Definition:

For the purposes of this invention, the following terms will have the meaning as specified therein:

“Pre-treated biomass” or “Pretreatment of biomass” used herein clears away physical and chemical barriers that make native biomass recalcitrant and exposes cellulose for better enzymatic hydrolysis. In most of the pretreatment, chemical (acid or alkali) and physical (high temperature or pressure) parameters are used individually or in mixed manner to remove barriers for enzymatic hydrolysis and improve the enzymatic digestibility.

“Detoxification” used herein is the process where the inhibitors (toxic compound such hydroxymethyl furfural, furfural, acetic acids, formic acids etc.) produced during the pretreatment process are removed or neutralized from pre-treated biomass by chemical, physical or biological process.

“Cellulase enzyme” used herein is a mixed form of enzyme which is mostly composed of exo-hydrolase, endo-hydrolase and beta-glucosidase. This enzyme was mostly produced from fungal sources. Cellulase breaks down the cellulose molecule into monosaccharide and shorter polysaccharides or oligosaccharides. In the present invention the cellulase enzyme is selected from commercial available cellulase enzymes which are suitable for the purposes. More particularly commercial available cellulase enzyme CTec3 is used in the present invention.

“Free sugar” used herein is the monomeric form of sugar which are produced from the lignocellulosic biomass during the pretreatment. Free sugar in this process composed of mainly glucose and xylose.

“C5 sugars” used herein C5 sugars represented for Xylose. “Free C5 sugar” used herein is sugar (mostly xylose) released from the hemicelluloses during the pretreatment and some part in enzymatic hydrolysis.

“C5 fermentation” used herein is Xylose fermentation into ethanol.

“C6 sugar” used herein represents glucose.

“C6 fermentation” used herein is Glucose fermentation into ethanol.

“Nutrient” used herein is Ammonium hydroxide and MgSO₄. Ammonium hydroxide used in this process has dual activity, it adjust the pH of the sulphuric acid (H₂SO₄) pretreated biomass and simultaneously converted to ammonium sulphate (ammonium ion (NH₄⁻) combined with free sulphates (SO₄⁻²) ions released from the sulphuric acid during the pretreatment. Ammonium sulphate ((NH₄)₂SO₄) acts as a nitrogen source for yeast during fermentation. Another salt MgSO₄ used in fermentation where, Mg⁺² act as an essential enzyme cofactor and act as key structural component of most biological pathways. During fermentation Mg⁺² plays a major role for proper functioning of fermenting enzymes in yeast.

The present invention discloses a method for production of ethanol from lignocellulosic biomass. In the present invention, free C5 sugar in pre-treated biomass is targeted first along with available low concentration of glucose for fermentation followed by enzymatic hydrolysis and C6 fermentation in sequential manner.

Xylan and Glucan are polymer of xylose and glucose respectively collectively called as holocellulose in lignocellulosic biomass. As per the physical property xylan and glucan are amorphous and crystalline in nature respectively. Due to the physical property, xylan gets breaks down to xylose when lignocellulosic biomass subjected to acid pretreatment but most of the glucan remain un-reacted. So in this process when the pretreated biomass is taken for fermentation, free form of xylose (30-35 g/L) (breakdown xylan) are present in the biomass which is targeted firstly by the co-fermenting microorganism for fermentation in presence of very less amount of glucose (<8-10 g/L, which is released during the pretreatment) at 30-35° C. The fermentation temperature is not adequate enough for the enzyme to breakdown of the glucan to glucose efficiently. So due to this the co-fermenting microorganism mostly targeted xylose (C5) sugar at the initial stage of fermentation.

The process, in accordance with the present invention, brings the C5 concentration about to dryness and brings down the total process time (both hydrolysis and fermentation) to 46 h which is about ⅓ of the conventional SHF (total process time 120 h which include 72 h Hydrolysis and 48 h Fermentation). Overall ethanol productivity is much higher than conventional SSCF process.

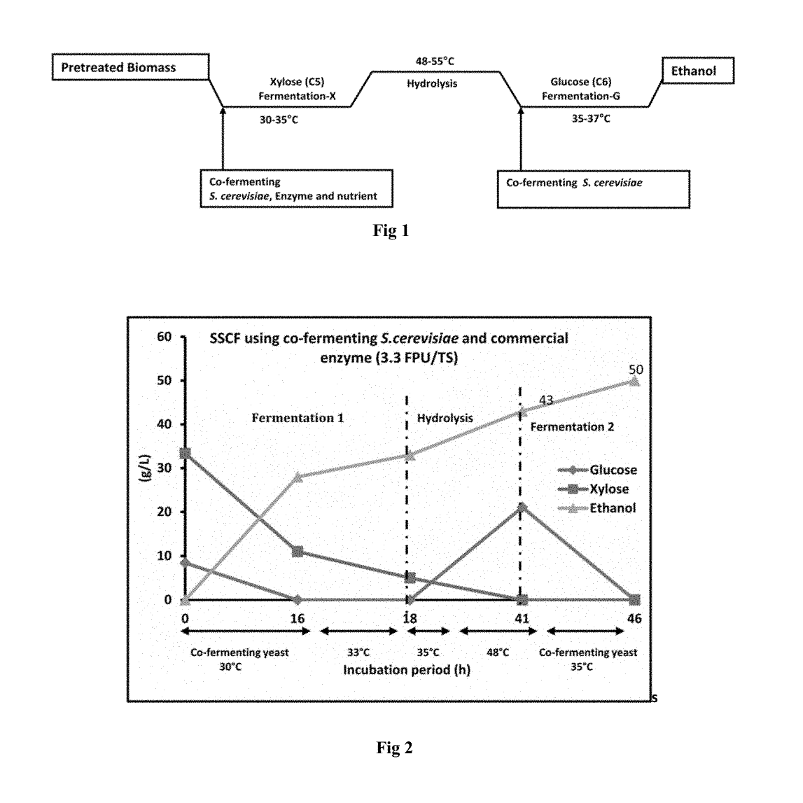

In accordance with the present invention, a method for production of ethanol from lignocellulosic biomass (see FIG. 1) is disclosed, comprising:
1. Addition of acid pre-treated biomass slurry without any detoxification in the fermenter;
2. The pH of the slurry was adjusted to 5-5.5 with aqueous ammonium solution. The pH adjusted slurry was fortified with 3-5 g/l MgSO₄, cellulase enzyme and co-fermenting Saccharomyces cerevisiae; the co-fermenting yeast is essential to use in this process to utilize both pentose and hexose sugar in the fermentation broth.
3. Appropriate water is added in to fermenter to adjust the final Total solid (TS) loading at 20% of biomass. The whole process is then incubated at 30° C.-35° C. for 16-20 hours for the fermentation with 200 rpm;
4. When the concentration of free xylose (free xylose is produced during the process of acid pretreatment and readily available for fermentation during fermentation process) in the slurry comes down to 3-5 g/l, the temperature of the process was slowly increased to 48-55° C. at ramping of 3 to 4° C. per 20-25 min., then the process is allowed to maintain about 48-55° C. temperature for 18-24 hours. After this incubation the system was allowed to cool down to temperature 35-37° C.;
5. A second dose of co-fermenting Saccharomyces cerevisiae is inoculated to the system for the second stage of fermentation. The second fermentation was stopped after 6 to 10 hours of fermentation.

Having described the basic aspects of the present invention, the following non-limiting examples illustrate specific embodiment thereof.

### EXAMPLE 1

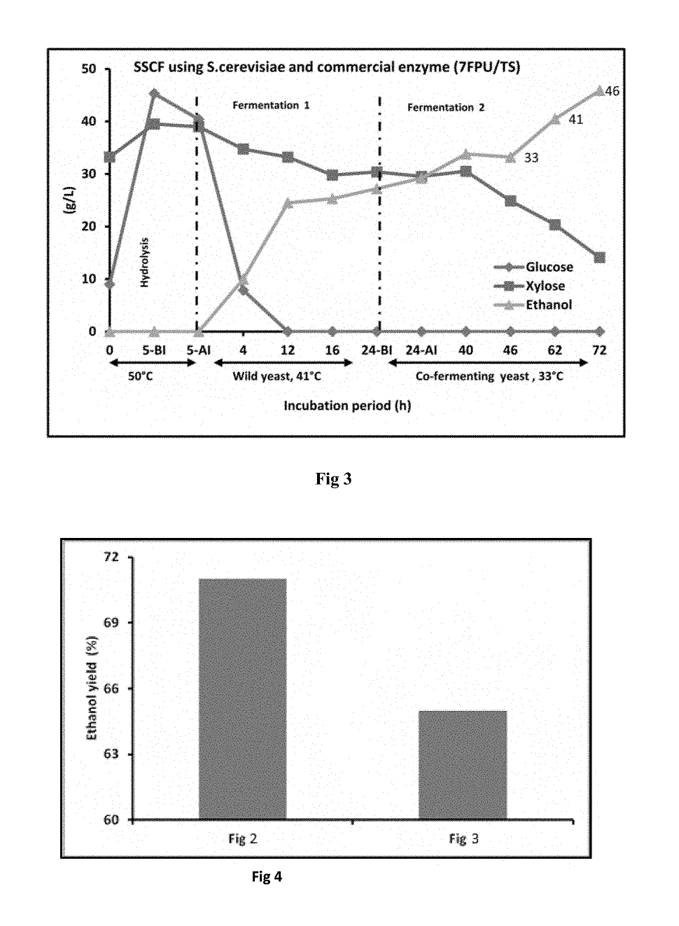

Pretreated biomass (slurry, TS approximately 24%) without any detoxification is introduced directly to the fermenter. The pH of the slurry was adjusted to 5.5 with aqueous ammonium solution (25% initial concentration). The pH adjusted slurry was fortified with 3 g/l MgSO₄, cellulase enzyme (Commercial enzyme, 3.3FPU/TS) and co-fermenting Saccharomyces cerevisiae (1 g dry cell biomass/litre, xylose and glucose utilizing yeast). Required amount of water was added to the process to adjust the final biomass concentration to 20%. The whole process was incubated at 30° C. for 16 h for the fermentation with 200 rpm. When the free xylose concentration in the slurry comes near to 6-7 g/l, the temperature of the process was increased to 33° C. and 35° C., incubated for 2 h in each temperature for better hydrolysis and fermentation. After that temperature increased to 48° C. This step mainly required for rapid releases of glucose sugar from cellulose which converted simultaneously with hydrolysis to ethanol by yeast biomass. As the temperature was reached at desired target the process was allowed to maintain the required temperature (48° C.) for 23 h for better enzymatic hydrolysis. After this incubation the system was allowed to cool down to temperature 35° C. A second dose of co-fermenting S. cerevisiae (1 g dry cell biomass/liter) was inoculated to the system for the second stage of fermentation. The second fermentation was stopped after 6 h of fermentation. This process took 46 h incubation including fermentation and enzymatic hydrolysis. The results of this experiment are represented by FIG. 2.

| | |
|---|---|
| Solid Loading in Fermentation | 20% |
| Mode of Fermentation | SSCF, Single yeast strain |
| | co-fermenting |
| | Saccharomyces cerevisiae |
| | (2 g/L (used in both fermentation |
| Enzyme loading (FPU/g) and Sources | 3.3, Commercial enzyme |
| Residual Xylose (g/L) | 1.30 |
| Ethanol Concentration (g/L) at 46 h | 50 |
| Ethanol Yield (%) | 71 |
| Specific Productivity (Q) g/L/h | 1.08 |

### EXAMPLE 2

Using conventional SSCF approach of ethanol production from pretreated biomass, saccharification at 50° C. for 5 h and followed by fermentation and hydrolysis at 41° C. by a moderately thermo tolerant wild yeast S. cerevisiae up to 24 h. After this fermentation another yeast co-fermenting S. cerevisiae was inoculated to the fermentation process. In this approach the xylose utilization after the glucose fermentation was comparatively slow as compare to the above process and about 10 g/l residual xylose was observed after 72 h. This process of fermentation brings the lower ethanol titer after the 72 h of fermentation using even higher enzyme dosage. The results of this experiment are represented by FIG. 3.

| | |
|---|---|
| Solid Loading in Fermentation | 20% |
| Mode of Fermentation | SSCF, 1^{st} wild type |
| | Saccharomyces cerevisiae (1 g/l), |
| | 2^{nd} co-fermenting |
| | Saccharomyces cerevisiae (1 g/l) |
| Enzyme loading (FPU/g) and Sources | 7, Commercial enzyme |
| Residual Xylose (g/L) | 9.98 |
| Ethanol Concentration (g/L) at 72 h | 46 |
| Ethanol Yield (%) | 65 |
| Specific Productivity (Q) g/L/h | 0.64 |

FIG. 4 represents the comparative ethanol yield (%) from Example 1 (Modified SSCF) and 2(Conventional SSCF process).

### REFERENCES

1. Krishnan, C., Sousa, L. D., Jin, M. J., Chang, L. P., Dale, B. E., Balan, V. 2010. Alkali-based AFEX Pretreatment for the conversion of sugarcane bagasse and cane leaf residues to ethanol. Biotechnology and Bioengineering, 107(3), 441-450.
2. Dien, B. S., Cotta, M. A., Jeffries, T. W. 2003. Bacteria engineered for fuel ethanol production: current status. Applied Microbiology and Biotechnology, 63(3), 258-266.
3. Taherzadeh, M. J., Karimi, K. 2007. Enzyme-based hydrolysis processes for ethanol from lignocellulosic materials: a review. Bioresources, 2(4), 707-738.
4. Lau, M. W., Dale, B. E. 2009. Cellulosic ethanol production from AFEX-treated corn stover using Saccharomyces cerevisiae 424A (LNH-ST). Proceedings of the National Academy of Sciences of the United States of America, 106(5), 1368-1373.

1. A process for production of ethanol from a lignocellulosic biomass comprising;
(i) adding slurry of a pre-treated lignocellulosic biomass comprising C5 and C6 sugars in a fermenter system;
(ii) selectively fermenting mainly C5 sugars by incubating the pretreated lignocellulosic biomass with a cellulase enzyme, co-fermenting microorganism and nutrient to obtain ethanol;
(iii) hydrolysing by heating the fermenter system to 48-55° C. for a period of 18 to 24 hours;
(iv) allowing the fermenter system to cool to a temperature of 35-37° C.; and
(v) selectively fermenting C6 sugars by inoculating the system with a second dose of co-fermenting microorganism to obtain ethanol.

2. The process as claimed in claim 1, wherein the fermentation of C5 sugar is carried out at temperature in the range of 30° C.-35° C. for 16-20 hours or any other temperature which favors fermentation over hydrolysis, when the xylose concentration is reduced to 6-7 g/l in fermentation broth the temperature of process is increased to 33 and 35° C. gradually and incubated at 2 h in each temperature for better hydrolysis and fermentation.

3. The process as claimed in claim 1, wherein the fermentation of C6 sugar is carried out at temperature in the range of 35 to 37° C. for 6 to 10 hours or any other temperature which favors fermentation over hydrolysis.

4. The process as claimed in claim 1, wherein the pre-treated biomass slurry is added in the fermenter system of step (i) without any detoxification.

5. The process as claimed in claim 1, additionally comprising adjusting pH of the slurry of step (i) to 5-5.5 with a pH adjuster.

6. The process as claimed in claim 5, wherein the pH adjuster is selected from aqueous ammonium hydroxide, NaOH, KOH, and CaCO₃ or substance which is alkaline in nature and increases pH.

7. The process as claimed in claim 1, wherein the nutrient is ammonium sulphate, MgSO₄ or any other magnesium or ammonium salts.

8. The process as claimed in claim 1, wherein the cellulase enzyme is from fungal origin, β-glucosidase along with other accessory enzyme, wherein:
(i) the cellulase enzyme of fungal origin is composed of cellobiohydrolase I and II; and
(ii) the other accessory enzymes is selected from xylanase, β-xyloxidase, arabinofuranosidase, and pectinse.

9. The process as claimed in claim 1, wherein the co-fermenting microorganism is selected from Saccharomyces cerevisiae, Pichia sp., Candida sp., and E. coli or any ethanogenic co-fermenting microorganism.

10. The process as claimed in claim 1, wherein the C5 sugar is selected from xylose and C6 sugar is selected from glucose.

11. The process as claimed in claim 1, wherein the lignocellulosic biomass is selected from straw, wheat straw, sugarcane bagasse, cotton stalk, barley stalk, bamboo or any agriculture residues which contain cellulose or hemicellulose or both.

12. A process for production of ethanol from a lignocellulosic biomass comprising:
(i) adding a pre-treated biomass slurry comprising C5 and C6 sugars without any detoxification in a fermenter system;
(ii) adjusting pH of the slurry of step (i) to 5-5.5 with aqueous ammonium solution to obtain a pH adjusted slurry;
(iii) fortifying the pH adjusted slurry with MgSO₄ in amount of 3-5 g/l, along with cellulase enzyme and co-fermenting microorganism;
(iv) adding water to the slurry of step (iii) to maintain 5 to 20 weight % Total solid (TS) in the slurry;
(v) incubating the slurry of step (iv) at 30° C.-35° C. for 16-20 hours for a selectively fermenting C5 sugars with 200-250 rpm when free xylose in the slurry to comes down to 6-7 g/l from 30 to 35 g/l;
(vi) hydrolysing by heating the fermenter system to 48-55° C. at ramping of 3 to 4° C. per 20-25 minutes, and then the process is allowed to maintain the temperature 48° C.-55° C. for 18-24 hours;
(vii) allowing the fermenter system to cool down to a temperature 35-37° C.; and
(viii) selectively fermenting C6 sugars by inoculating the system with a second dose of co-fermenting microorganism to obtain ethanol.
